(19) United States
(12) Patent Application Publication
(10) Pub. No.: US 2014/0356869 A1
(43) Pub. Date: Dec. 4, 2014

(54) SINGLE NUCLEOTIDE POLYMORPHISMS ASSOCIATED WITH DIETARY WEIGHT LOSS

(71) Applicant: DSM IP ASSETS B.V., The Heerlen (NL)

(72) Inventor: Thorklid Sorensen, Copenhagen (DK)

(21) Appl. No.: 14/361,084

(22) PCT Filed: Nov. 22, 2012
(86) PCT No.: PCT/EP2012/073330

§ 371 Date: May 28, 2014

(30) Foreign Application Priority Data
Nov. 28, 2011 (EP) 11190945.3

(51) Int. Cl. C12Q 1/68

(52) U.S. Cl. CPC C12Q 1/6883; C12Q 2600/156; C12Q 2600/112
USPC 435/6.11

(57) ABSTRACT

The present invention relates to genetic polymorphisms associated with obesity and obesity-related phenotypes and their use in predicting if an individual looses weight during a dietary weight loss intervention program.

### FIELD OF THE INVENTION

The present invention is in the field of obesity. More in particular it relates to genetic polymorphisms and their effect on dietary weight loss intervention programs. Moreover, the present invention pertains to genetic tests and methods using the polymorphisms, particularly methods to predict an obese individual's likelihood to loose weight in conjunction with a specific dietary weight loss intervention program.

### BACKGROUND OF THE INVENTION

Obesity is a worldwide epidemic found across all age groups. Especially in industrialized countries, it has increased at a fast rate over the past two decades and is now a worldwide leading public health problem. For example, while in 1996 26% adult Americans were overweight and 10% severely so, currently more than 65% are overweight, with nearly 31% meeting the criteria for obesity. As obesity portends an epidemic of related chronic diseases such as type-2 diabetes, hypertension and cardiovascular events, people with obesity especially people with extreme obesity are at risk for many health problems. The economic cost attributable to obesity in the United States alone has been estimated to be as high as $100 billion per year and includes not only direct health care costs but also the cost of lost productivity in affected individuals.

While diet and lifestyle contribute to obesity and the trend of decreased physical activity and increased caloric intake is probably responsible for the recent rise in obesity, it is important to understand that genetics plays a key role. Each individual's genetic background remains an important determinant of susceptibility to obesity. For instance, half of the population variation in body mass index (BMI), a common measure of obesity, is determined by inherited factors.

Many studies have reported that common genetic variants, usually single nucleotide polymorphisms (SNPs), are associated with an increased risk for obesity. Two approaches have been used to date to find these variants, linkage analysis and association studies. Although on the basis of linkage studies some regions have been repeatedly implicated to play a role in obesity, no genes have been found in these regions that have been seen to contribute to the disease. By using association studies several associations between obesity or obesity-related traits and common genetic variants have been reported. Unfortunately, many of the reported associations have not been consistently replicated.

A gene which has recently been reproducibly associated with diabetes is the Transcription Factor Activating enhancer binding Protein 2 beta (TFAP2B) gene. It is preferably expressed in adipose tissue. Overexpression of TFAP2B leads to increase glucose uptake and thereby triglyceride accumulation and insulin resistance in adipocytes. Abnormal secretion of adipocytokines promotes diabetes and insulin resistance. TFAP2B expression has shown an inverse correlation with leptin and adiponectin expression, but a positive correlation with expression of inflammatory adipokines, IL-6 and MCP-1, in adipose tissue.

Altering dietary habits is the cornerstone of weight loss intervention programs for overweight and obese patients. One diet may not be optimal for weight loss in all overweight or obese individuals, so future dietary guidance could become more individualized to increase success rates for weight loss. Despite the increasing knowledge of loci and genes associated with obesity and obesity-related traits, no useful genetic variants exist on the basis of which dietary weight loss intervention programs can be tailored for overweight or obese individuals. Studies investigating genetic variants and diet in combination have so far failed to find strong effects of genetic variation on the dietary effect on weight loss. Identification of such genetic variants will contribute to an increased understanding of obesity, and may lead to more individualized treatment of obesity, e.g. personalized nutrition for weight loss. Importantly, promising results from a study needs replication in other studies for strengthening the evidence of the observed effects. The present invention is based on strong results for a gene-diet interaction in one study, further supported by results in another study.

### SUMMARY OF THE INVENTION

It was found in accordance with the present invention that markers exist that are associated with the likelihood that an individual, such as an overweight or obese individual, loose weight depending on the energy restricted diet used. The term “associated with” in connection with the relationship between a genetic characteristic, e.g., a marker, allelic variant, haplotype, or polymorphism, and a trait means that there is a statistically significant level of relatedness between them based on any generally accepted statistical measure of relatedness. Those skilled in the art are familiar with selecting an appropriate statistical measure for a particular experimental situation or data set. Accordingly, the present invention is directed to methods wherein use is made of the genetic characteristics. The markers can for instance be found in genes associated with overweight, obesity or obesity-related traits. The resulting information can be used to classify individuals such as overweight or obese individuals based on their genetic tendency to loose weight depending on the types of energy restricted diet they consume. This will help professionals in the field of weight management to improve targeting these individuals with appropriate (nutritional) advice regarding their weight management. As a result thereof, the success rate of dietary weight loss intervention programs will increase.

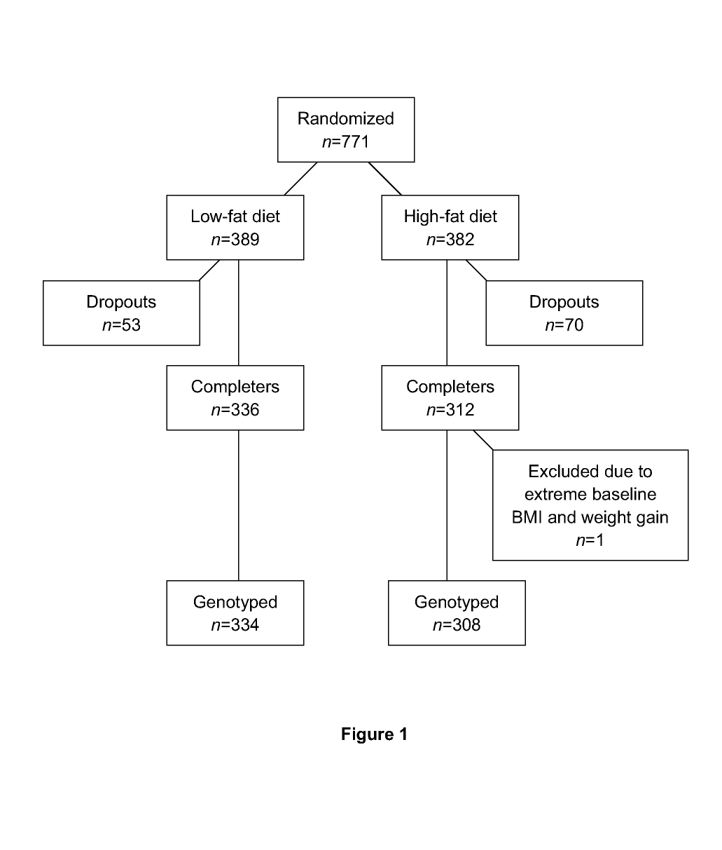

FIG. 1. Participant flow in NUGENOB study.

### DETAILED DESCRIPTION OF THE INVENTION

The invention is based on the surprising finding that obese individuals carrying the AA genotype of the SNP rs987237 located in the TFAP2B gene, lose more weight on an energy-restricted diet low in fat (high in carbohydrates) than on an iso-caloric diet high in fat. In contrast, individuals with the AG or GG variant, showed greater weight loss effects on the high-fat diet. This surprising finding does not result from a single study, but importantly, has also been confirmed in another weight loss trial of obese participants with similar characteristics (BMI, age, geographical spread etc) to those in the first analysis. The TFAP2B polymorphism of the present invention may thus be useful in predicting the outcome of weight loss by diets of different proportions of fat and carbohydrates. The polymorphism may be part of a haplotype which may have an association link with the likelihood of an individual to loose weight in energy restricted weight loss intervention program.

As used herein, “haplotype” refers to a set of alleles found at linked polymorphic sites on a single chromosome. The linked sites may include part of a gene, an entire gene, several genes, or a region devoid of genes (but which perhaps contains a DNA sequence that regulates the function of nearby genes). The haplotype preserves information about the phase of the polymorphic nucleotides, that is, which set of variances were inherited from one parent (and are therefore on one chromosome) and which from the other. In a preferred embodiment the programs comprise dietary intervention either alone or as a major component. Next to suitable diets, i.e. personalized diets based on the genetic profile of an individual, weight loss intervention programs may however also include other components such as e.g. drug treatment, surgical treatment e.g. liposuction, behavioural therapy, increase in physical activity and dietary supplement treatment.

In view of the fact that obese individuals that carry two A alleles of the SNP rs987237 have more difficulty in loosing weight on energy restricted diet comprising a high fat/low carbohydrate diet than a low fat/high carbohydrate diet, the identification of an obese individual carrying two A alleles can help weight management professionals to design suitable dietary weight loss intervention programs for these individuals.

Therefore, in an aspect, the invention relates to the use of at least one genetic marker such as a polymorphism, e.g. a SNP, for determining what type of diet an individual may be given in order to most efficiently loose weight. In other words, the data provided herein show that a correlation, association, linkage or other relation between a specific marker and the capacity to loose weight when given a specific type of diet can be established. The marker determines the likelihood that an individual looses weight under a specific diet. Diets used in dietary weight loss intervention programs designed to treat individuals are well known to the skilled person. Preferred diets in the light of the present invention include but are not limited to high fat/low carbohydrate diets or low fat/high carbohydrate diets. The high fat/low carbohydrate or low fat/high carbohydrate diets may be hypo-energetic diets (also called hypo-caloric diets). In an embodiment the individual is overweight or obese which is characterized by a BMI of at least 30 kg/m². An “individual” as used in the present application refers to a vertebrate, preferably a mammal, more preferably an animal such as a domestic animal (e.g. a dog or cat), and most preferably a human.

An “overweight individual”, as used herein, refers to an individual fulfilling the normal definition of overweight individual as defined by the medical knowledge at the time of diagnosis. Useful criteria for defining an individual as overweight include, but are not limited to, a body mass index (BMI) of 25-29.9, male individual with a waist measurement greater than 40 inches (102 cm), female individual with a waist measurement greater than 35 inches (88 cm), and all individuals with a waist-to-hip ratio of 1.0 or higher. An “obese individual”, as used herein, refers to an individual fulfilling the normal definition of obese individuals as defined by the medical knowledge at the time of diagnosis. Useful criteria for defining an individual as obese include, but are not limited to, a body mass index (BMI) of 30 or higher.

A “hypo-energetic (hypo-caloric) diet” as used herein means a diet wherein the daily energy intake is less than the daily energy requirement, e.g. a diet with an energy deficiency of at least 100, 200, 300, 400, 600, 800, 1000, 1200, 1500 or 2000 kcal/day. “High-fat” diets as used herein means diets having at least 30%, preferably at least 40%, more preferably 40-45% of energy intake derived from fat. “Low-fat” diets as used herein means diets having less than 30%, preferably less than 25%, more preferably 20-25% of energy from fat. “Low carbohydrate” diets as used herein means diets having less than 50%, preferably less than 45%, more preferably 40-45% of energy from carbohydrate. “High carbohydrate” diets as used herein means diets having at least 50%, preferably at least 60%, more preferably 60-65% of energy from carbohydrate. The diets may further contain other components such as e.g. proteins. The diets may have e.g. 15% of energy from proteins. Preferably, the individuals on the dietary intervention program do not consume alcohol. Where exclusion of alcohol is not possible, intake should be minimal, with an upper limit of two glasses (2×150 ml) in total. Energy from alcohol should be subtracted from total energy intake and thereafter macronutrient intake should be calculated on the remaining energy. Where possible, viscous soluble fibres should be avoided in the diets, since they are thought to have the greatest impact on glucose and lipid metabolism (e.g. oats and guar gum). Furthermore, it may be attempted to standardise other sources of soluble fibre within the diets (e.g. fruit and vegetables, especially legumes). Individuals participating in dietary intervention programs may be encouraged to consume equal amounts of polyunsaturated, monounsaturated and saturated fats by ensuring incorporation of olive oil (or equivalent) and sunflower oil (or equivalent) into each day's choices (in addition to saturated fat predominately from meat and dairy products). They may avoid using food products including specialist margarines which contain added plant sterols, omega-3 fatty acids or soy compounds, and soy based products. Furthermore, they may be encouraged to consume oily fish at least once a week within the fat restriction of the diet and they may attempt to maintain comparable ratios of simple sugars to complex carbohydrates. Individuals who are already taking vitamin and mineral supplementation before starting the dietary intervention program may continue taking the same dose throughout the program and this intake may be included in the intake analysis.

Therefore, in another embodiment of the invention the marker is present in a locus, gene or gene cluster associated with an obesity-related phenotype. As used herein, “phenotype” refers to any observable or otherwise measurable physiological, morphological, biological, biochemical or clinical characteristic of an individual. Of course, a combination of markers can be used in the methods, kits, uses, etc of the present invention. The markers may be present in coding (exons) but may also be present in non-coding regions (intron and intergenic regions). They may be present in different genes e.g. one marker in the TFAP2B gene (e.g. SNP rs987237) and another marker in e.g. the MC4R gene. If more than one marker is used, the markers may be in linkage disequilibrium with one another, preferably in non-tight linkage disequilibrium. “Linkage disequilibrium” or “allelic association” means the preferential association of a particular allele or genetic marker with a specific allele or genetic marker at a nearby chromosomal location more frequently than expected by chance for any particular allele frequency in the population. Linkage disequilibrium may result from natural selection of certain combination of alleles or because an allele has been introduced into a population too recently to have reached equilibrium (random association) between linked alleles. A marker in linkage disequilibrium with disease predisposing variants can be particularly useful in detecting susceptibility to disease (or association with sub-clinical phenotypes), notwithstanding that the marker does not cause the disease. Methods to determine linkage disequilibrium are well known to the skilled artisan. The present invention thus also pertains to methods and uses comprising determining in vitro the genotype of the SNP rs987237, and/or at least one other SNP in DNA taken from an individual. This other SNP may be in linkage disequilibrium with SNP rs987237.

Obesity-related phenotypes include but are not limited to body weight, BMI, percent fat mass (FM), percent fat-free mass (FFM), waist circumference (WC), insulin secretion (HOMA-β) insulin resistance (HOMA-IR), fasting energy expenditure (EE), serum triglycerides, cholesterol, and glucose, to name just a few. Genes associated with these phenotypes have been found (see Obesity: Genomics and postgenomics, Eds: Clement and Sørensen, Informa Healthcase, first edition, 2007). In a preferred embodiment the marker, e.g. SNP, is present in the TFAP2B gene. In a preferred embodiment the marker is the SNP rs987237. SNP rs987237, located in the TFAP2B gene, has been found consistently associated with BMI in white Europeans. Each additional G allele is related to 0.13 kg/m² higher BMI, so an individual of the GG genotype has on average 0.26 kg/m² higher BMI than an individual with the AA genotype. It is to be understood that any marker that is in linkage disequilibrium with rs987237 can also be used in the various aspects and embodiment of the present invention. These markers do not necessarily have to be present in the same locus, gene or gene cluster. They may be part of other more distant genes. However, they should be in linkage. “Linkage” describes the tendency of genes, alleles, loci or genetic markers to be inherited together as a result of their location on the same chromosome, and can be measured by percent recombination between the two genes, alleles, loci or genetic markers that are physically-linked on the same chromosome. Loci occurring within 50 centimorgan of each other are linked. Some linked markers occur within the same gene or gene cluster.

In a further aspect, the invention pertains to the use of at least one marker for determining which diet will result in the most successful weight loss in an individual. On the basis of an association between a genetic marker and a diet, in relation to weight loss, a suitable diet can be recommended to an individual and/or added to an individual's food or diet. Preferably, the marker is a genetic marker such as a polymorphism, e.g. a SNP.

As used herein “polymorphism” refers to DNA sequence variation in the cellular genome of an individual, typically with a population frequency of more than 1%. A polymorphic marker or site is the locus at which genetic variation occurs. Preferred markers have at least two alleles, each occurring at frequency of greater than 1%, and more preferably greater than 10% or 20% of a selected population. A polymorphic locus may be as small as one base pair. Polymorphic markers include restriction fragment length polymorphisms, variable number of tandem repeats, hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wild-type form. Diploid organisms may be homozygous or heterozygous for allelic forms. A SNP occurs at a polymorphic site occupied by a single nucleotide. A SNP usually arises due to substitution of one nucleotide for another at the polymorphic site, but it can also arise from an insertion or deletion of a nucleotide relative to a reference allele.

In yet another embodiment the invention relates to a method for assessing whether an individual will loose more weight when consuming a low fat/high carbohydrate diet or when consuming a high fat/low carbohydrate diet, the method comprising the step of determining the genotype of the single nucleotide polymorphism rs987237 in the TFAP2B gene, wherein:
(i) the presence of two A allelic forms of the SNP rs987237 is indicative of an increased likelihood that the individual looses more weight on a low fat/high carbohydrate diet compared to a high fat/low carbohydrate diet and,
(ii) the presence of either AG or GG allelic forms of the SNP rs987237 is indicative of an increased likelihood that the individual looses more weight on a high fat/low carbohydrate diet compared to a low fat/high carbohydrate diet.

In a preferred embodiment the individual is overweight or obese. In a further embodiment the individual is a white European. In a further embodiment the high fat/low carbohydrate or low fat/high carbohydrate diet is a hypo-energetic diet.

In another embodiment the present invention also provides a method for predicting the likelihood that an individual will loose weight during a dietary weight loss intervention program, the method comprising the steps of a) obtaining a biological sample comprising nucleic acid of the individual, and b) genotyping the nucleic acid for the single nucleotide polymorphism (SNP) rs987237 in the TFAP2B gene, wherein

(i) the presence of two A allelic forms of the SNP rs987237 is indicative of an increased likelihood that the individual looses more weight on a low fat/high carbohydrate diet compared to a high fat/low carbohydrate diet and,

(ii) the presence of AG or GG allelic forms of the SNP rs987237 is indicative of an increased likelihood that the individual looses more weight on a high fat/low carbohydrate diet compared to a low fat/high carbohydrate diet.

In the methods and uses of the present invention the occurrence of the A/G allelic form of the SNP rs987237 may be assessed by contacting a nucleic acid derived from the genome of an individual with a first oligonucleotide that anneals with higher stringency with the A allelic form of the polymorphism than with the G allelic form of the polymorphism and assessing annealing of the first oligonucleotide and the nucleic acid, whereby annealing of the first oligonucleotide and the nucleic acid is an indication that the genome of the individual comprises the A allelic form of the polymorphism. The method may be extended by assessing the occurrence of the G allelic form of the polymorphism by contacting the nucleic acid with a second oligonucleotide that anneals with higher stringency with the G allelic form of the polymorphism than with the A allelic form of the polymorphism and assessing annealing of the second oligonucleotide and the nucleic acid, whereby annealing of the second oligonucleotide and the nucleic acid is an indication that at least one allele of the TFAP2B gene in the genome of the individual does not comprise the A allelic form of the polymorphism. The first and second oligonucleotides may be attached to a support. It may be the same support.

“Biological sample” as used in the present invention encompasses a variety of sample types that can be used as source material for isolating nucleic acids. They include, but are not limited to, solid materials (e.g., tissue, tissue cultures or cells derived there from and the progeny thereof, hair follicle samples, biopsy specimens, buccal cells provided by a swab, skin and nose samples) and biological fluids (e.g. urine, faecal material, blood, semen, amniotic fluid, tears, saliva, sputum, sweat, mouth wash). Any biological sample from a human individual comprising even one cell comprising nucleic acid can be used in the methods of the present invention. The term also includes samples that have been manipulated in any way after their procurement, such as by treatment with reagents, solubilisation, or enrichment for certain components, such as proteins or polynucleotides. The methods and uses of the present invention are preferably conducted on a sample that has previously been removed from the individual and do preferably not involve diagnosis practised on the human body.

Nucleic acid molecules as used herein refers to polymeric forms of nucleotides and includes both sense and antisense strands of RNA, cDNA, genomic DNA, and synthetic forms and mixed polymers of the above, with genomic DNA being preferred. A nucleotide refers to a ribonucleotide, deoxynucleotide or a modified form of either type of nucleotide. The term also includes single- and double-stranded forms of DNA. In addition, a polynucleotide may include either or both naturally-occurring and modified nucleotides linked together by naturally-occurring and/or non-naturally occurring nucleotide linkages. The nucleic acid molecules may be modified chemically or biochemically or may contain non-natural or derivatized nucleotide bases, as will be readily appreciated by those of skill in the art. Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule. A reference to a nucleic acid sequence encompasses its complement unless otherwise specified. Thus, a reference to a nucleic acid molecule having a particular sequence should be understood to encompass its complementary strand, with its complementary sequence. The complementary strand is also useful, e.g., for antisense therapy, hybridization probes and PCR primers.

Nucleic acids can be isolated from a particular biological sample using any of a number of procedures, which are well-known in the art, the particular isolation procedure chosen being appropriate for the particular biological sample. Methods of isolating and analyzing nucleic acid variants as described above are well known to one skilled in the art and can be found, for example in the Molecular Cloning: A Laboratory Manual, 3rd Ed., Sambrook and Russel, Cold Spring Harbor Laboratory Press, 2001 and Current Protocols in Molecular Biology Volumes I-III, 4^{th} edition, Ausubel et al., John Wiley and Sons, 1995. Many of the methods require amplification of nucleic acid from target samples. This can be accomplished by techniques such as e.g. PCR, ligase chain reaction, nucleic acid based sequence amplification, self-sustained sequence replication and transcription amplification. Genetic markers such as the SNPs can be detected from the isolated nucleic acids using techniques including DNA hybridization methods (e.g. Southern Blotting, FISH), direct sequencing with radioactively, enzymatically, luminescently or fluorescently labelled primers (manually or automated), restriction fragment length polymorphism (RFLP) analysis, heteroduplex analysis, single strand conformational polymorphism (SSCP) analysis, denaturing gradient gel electrophoresis (DGGE), temperature gradient gel electrophoresis (TGGE), use of linked genetic markers, mass spectrometry e.g. MALDI-TOF, and chemical cleavage analysis to name just a few. Of course DNA MicroArray technology suitable for detecting genetic markers such as SNPs can also be used. All methods are explained in detail, for example, in the Molecular Cloning: A Laboratory Manual, 3rd Ed., Sambrook and Russel, Cold Spring Harbor Laboratory Press, 2001.

Primers used may be oligonucleotides hybridizing specifically with one allele. They are called allele-specific oligonucleotides. In the allele-specific PCR methodology, a target DNA is preferentially amplified only if it is completely complementary to the 3′-terminus of a specific PCR amplification primer. The 3′-terminus of the primer is designed so as to terminate at, or within one or two nucleotides of a known mutation site within the target DNA to which it possesses a complementary sequence. Under the appropriate reaction conditions, the target DNA is not amplified if there is a single nucleotide mismatch (e.g., a nucleotide substitution caused by a mutation) or a small deletion or insertion, at the 3′-terminus of the primer. Accordingly, allele-specific PCR may be utilized to detect either the presence or absence of (at least) a single nucleotide mismatch between the primer sequence (which is complementary to a pre-selected target sequence) and a nucleic acid within the sample. Amplification of the target sequence is indicative of a lack of even a single mismatched nucleotide. The markers in the present invention are preferably analyzed using methods amenable for automation. Primer extension analysis can be performed using any method known to one skilled in the art. Oligonucleotides, probes and/or primers may be naturally occurring or synthetic, but are typically prepared by synthetic means. They may be immobilized on a solid support. For instance, oligonucleotides, probes and/or primers as described herein can be used as a DNA chip. The chip may contain a primer corresponding to a single allelic form of a marker but may also contain a primer corresponding to both allelic forms of a marker. It may even comprise primers for different markers. The appropriate length of an oligonucleotide, probe and/or primer depends on its intended use but typically ranges from 10 to 75, preferably 15 to 40 nucleotides. Short primer molecules generally require cooler temperatures to form sufficiently stable hybrid complexes with the template. A primer need not reflect the exact sequence of the template but must be sufficiently complementary to hybridize with a template. Conditions suitable for hybridization are generally known in the art and will be apparent to the skilled artisan. A non-limiting example of stringent hybridization conditions is hybridization in 6× sodium chloride/sodium citrate (SSC) at about 45° C., followed by one or more washes in 0.2×SSC, 0.1% SDS at 50-65° C. Stringent conditions can for instance be found in Molecular Cloning: A Laboratory Manual, 3rd Ed., Sambrook and Russel, Cold Spring Harbor Laboratory Press, 2001 and Current Protocols in Molecular Biology, John Wiley & Sons, N.Y. (1989), 6.3.1-6.3.6. The term “primer site” refers to the area of the target DNA to which a primer hybridizes. The term “primer pair” means a set of primers including a 5′-upstream primer that hybridizes with the 5′-end of the DNA sequence to be amplified and a 3′-downstream primer that hybridizes with the complement of the 3′-end of the sequence to be amplified.

As used herein, “genotype” refers to the genetic constitution of an individual. More specifically, “genotyping” as used herein refers to the analysis of DNA in a sample obtained from a subject to determine the DNA sequence in a specific region of the genome, e.g. a locus that influences a trait. It may refer to the determination of DNA sequence at one or more polymorphic sites and/or determination of allelic patterns of an individual. The genotyping may be performed using a micro-array or a multi-well plate in for instance a laboratory or hospital. It may thus involve the use of a gene/DNA chip or a strip or solid surface comprising one or more nucleic acid molecules.

In another embodiment, the present invention relates to a method for making a dietary weight-loss intervention program for an individual, the method comprising the steps of:

(i) identifying the individual's genotype for the single nucleotide polymorphism rs987237 in the TFAP2B gene,

(ii) using a low fat/high carbohydrate diet in the making of a dietary weight-loss intervention program suitable for treating an individual which has been identified as having two A allelic forms of the single nucleotide polymorphism rs987237 in the TFAP2B gene,

and using a high fat/low carbohydrate diet in the making of a dietary weight-loss intervention program suitable for treating an individual which has been identified as having AG or GG allelic forms of the single nucleotide polymorphism rs987237 in the TFAP2B gene.

In a further aspect, the invention pertains to a method of assessing the desirability of treating an individual with a low fat/high carbohydrate or a high fat/low carbohydrate diet, the method comprising assessing occurrence of the SNP rs987237 in the TFAP2B gene of the individual, wherein occurrence of two A allelic forms of the SNP is an indication that it is more desirable to treat the individual with a low fat/high carbohydrate diet than with a high fat/low carbohydrate diet and wherein the presence of AG or GG allelic forms of the SNP is an indication that it is more desirable to treat the individual with a high fat/low carbohydrate diet than with a low fat/high carbohydrate diet.

In other words, the present invention provides a method of assessing the advisability that a individual should employ a dietary weight loss intervention program comprising either a high fat/low carbohydrate diet or a low fat/high carbohydrate diet, the method comprising assessing the occurrence in the genome of the individual of the AA allelic form of SNP rs987237, whereby presence of this form of the polymorphism is an indication that the individual should employ low fat/high carbohydrate diet while presence of either of AG or GG allelic forms of the polymorphism is an indication that the individual should employ high fat/low carbohydrate diet.

The invention further provides a method of assessing the desirability of supplementing the food of an individual with either a high fat/low carbohydrate diet or a low fat/high carbohydrate diet, the method comprising assessing occurrence in the genome of the individual of the two A allelic forms of the SNP rs987237, whereby occurrence of a AA polymorphism is an indication that it is more desirable to supplement the individual's food with a low fat/high carbohydrate diet than that of an individual whose genome comprises one or two G allelic forms of the polymorphism. In an individual wherein the AG or GG allelic form is present, it is more desirable to supplement the individual's food with a high fat/low carbohydrate diet than that of an individual whose genome does comprise an AA allelic form of the polymorphism.

The invention is also directed to a method of determining whether an individual is a suitable candidate for a low fat/high carbohydrate or a high fat/low carbohydrate dietary weight loss intervention program, the method comprising the steps of genotyping the SNP rs987237 in the TFAP2B gene in a nucleic acid sample of the individual, wherein the occurrence of a AA allelic form of the SNP is indicative of the individual being a suitable candidate for a low fat/high carbohydrate dietary weight loss intervention program and wherein the presence of the AG or GG allelic form of the SNP is indicative of the individual being a suitable candidate for a high fat/low carbohydrate dietary weight loss intervention program.

In yet a further aspect the invention provides a method of determining whether an individual has an increased predisposition to loose weight in a dietary weight loss intervention program, the method comprising the step of a) isolating from the individual a nucleic acid comprising the single nucleotide polymorphism (SNP) rs987237, and b) determining the allelic form of the SNP, wherein the presence of two A allelic forms of the SNP indicates that the individual has an increased likelihood to loose weight on a low fat/high carbohydrate diet compared to a high fat/low carbohydrate diet and wherein the presence of AG or GG allelic forms of the SNP indicates that the individual has an increased likelihood to loose weight on a high fat/low carbohydrate diet compared to a low fat/high carbohydrate diet.

In another aspect the invention relates to a method of assessing the advisability that an individual should employ a high fat/low carbohydrate diet or a low fat/high carbohydrate diet, the method comprising the step of assessing occurrence in the individual's genome of the SNP rs987237, whereby occurrence of two A allelic forms of the SNP is an indication that the individual should employ a low fat/high carbohydrate diet and presence of AG or GG allelic forms of the SNP is an indication that the individual should employ a high fat/low carbohydrate diet.

In the methods of the invention the high fat/low carbohydrate or low fat/high carbohydrate diet may be a hypo-energetic diet.

The invention further relates to a method of correlating a specific allelic form of a SNP in a gene of an individual with the increased likelihood of the individual to succeed in a dietary weight loss intervention program, comprising a) identifying an individual (individual A) having successfully lost weight in a dietary weight loss intervention program, b) determining the allelic form of the SNP in the gene of individual A, c) comparing the allelic form of the SNP in the gene of individual A with the allelic form of the SNP in the gene of an individual having failed to loose weight in a dietary weight loss intervention program (individual B), and d) if the allelic form of the SNP differs between individual A and individual B, correlating the specific allelic form of the SNP in the gene of individual A with an increased likelihood of an individual to complete a dietary weight loss intervention program.

In the methods of the invention the individual can be an overweight or obese individual. In a further embodiment the individual is a white European.

The steps of obtaining a sample from an individual, determining the genotype of a SNP, e.g. SNP rs987237 in the TFAP2B gene, and linking the genotype of the SNP to a specific type of diet or dietary weight intervention program that is more or less suitable for the individual can be done by one party, however, the steps can also be performed by two or even more distinct parties.

Another aspect of the invention is directed to a kit for use in a method or use of the present invention. The kit comprises at least one primer or primer pair suitable for determining (or being associated with) the likelihood that an individual such as an overweight or obese individual loses weight in a dietary weight loss intervention program, more in particular a dietary component thereof such as a diet. In an embodiment the invention is directed to a kit for use in a method or use according to the invention, the kit comprising at least one primer or primer pair for genotyping a marker in a gene or locus associated with obesity or an obesity-associated phenotype such as the TFAP2B gene. Preferably, the marker is SNP rs987237 in the TFAP2B gene. The primers may be suitable for nucleic acid sequence amplification. Often the kits contain one or more primers or primer pairs hybridizing to different forms of a polymorphism, e.g. a primer or primer pair capable of hybridizing to the A allelic form of SNP rs987237 and a primer or primer pair capable of hybridizing to the G allelic form of SNP rs987237. Moreover, kits according to the invention may comprise instructions explaining correlation of the genotype to increased likelihood of loosing weight when following a dietary weight loss intervention program such as a high fat/low carbohydrate diet or a low fat/high carbohydrate diet. In case the marker is SNP rs987237, the instructions may explain that detection of two A allelic forms of the SNP is indicative of the individual as being more likely to respond to (more susceptible for) a low fat/high carbohydrate dietary weight loss intervention program than to a high fat/low carbohydrate dietary weight loss intervention program and that detection of AG or GG allelic forms of the SNP is indicative of that individual being more likely to respond to (more susceptible for) a high fat/low carbohydrate dietary weight loss intervention program than to a low fat/high carbohydrate dietary weight loss intervention program. Optional additional components of the kit include, for example, restriction enzymes, reverse transcriptase or polymerase, a positive control, a negative control, at least a further primer pair suitable for detecting (other) markers, appropriate buffers for reverse transcription, PCR and/or hybridization reactions, means used to label and nucleotide mix for the PCR reaction. The kits of the invention may thus also comprise one or more primers, primer pairs, probes and/or oligonucleotides suitable for detecting markers such as SNPs which is/are in linkage disequilibrium with SNP rs987237. In addition, a kit according to the present invention may contain instructions for carrying out the methods as well as a listing of the obesity-associated alleles and haplotypes relevant in view of the present invention. The components of the kit may be either in dry form in a tube or a vial or dissolved in an appropriate buffer.

The present invention employs, unless otherwise indicated, conventional (recombinant) techniques of molecular biology, immunology, microbiology, biochemistry and cell biology which are well within the skill of a person skilled in the art. All publications and references cited in the present application are incorporated by reference in their entirety for any purpose.

Furthermore, in yet another embodiment the present invention relates to the use of a low fat/high carbohydrate diet in an individual which has been identified as having two A allelic forms of the SNP rs987237 in the TFAP2B gene. Moreover, in another embodiment the present invention relates to the use of a high fat/low carbohydrate diet in an individual which has been identified as having AG or GG allelic forms of the SNP rs987237 in the TFAP2B gene. The specific diet can be used in a dietary weight loss intervention program for the individual. In the present invention is also encompassed the use of a low fat/high carbohydrate diet in the manufacture of a medicament for the treatment and/or prevention of obesity in an individual which has been identified as having two A allelic forms of the SNP rs987237 in the TFAP2B gene. It also relates to the use of a high fat/low carbohydrate diet in the manufacture of a medicament for the treatment and/or prevention of obesity in an individual which has been identified as having AG or GG allelic forms of the SNP rs987237 in the TFAP2B gene. In a further aspect the invention relates to the use of a low fat/high carbohydrate diet or high fat/low carbohydrate diet in the making of a dietary weight intervention program suitable for treating an individual which has been identified as having two A allelic forms of the SNP rs987237 in the TFAP2B gene or AG and GG allelic form of the SNP rs987237 in the TFAP2B gene, respectively. In an embodiment the individual is obese.

The invention further relates to the use of the single nucleotide polymorphism rs987237 in the TFAP2B gene for selecting a diet an individual should be provided to loose weight during a weight loss intervention program.

In another embodiment the present invention also relates to computer systems and computer readable media for storing data according to the present invention. Computer readable media mean media that can be read and accessed directly by a computer including but not being limited to magnetic storage media e.g. floppy discs, hard disc storage media and magnetic tapes; optical storage media e.g. CD-ROM; electrical storage media e.g. RAM and ROM; and hybrids of these categories e.g. magnetic/optical storage media. The data can be stored in one or more databases and include information relating to markers e.g. SNPs such as SNP rs987237 suitable for determining the likelihood that an individual looses weight during a dietary weight loss intervention program. The databases may further include information regarding the nature of the marker (e.g. the base occupying a polymorphic position in a reference allele as well as in a non-reference allele), the location of the marker (e.g. by reference to for example a chromosome or distance to known markers within the chromosome), the level of association of the marker with obesity, the frequency of the marker in the population or a subpopulation, the association of the marker with other markers as well as all relevant information about the other markers. It may also include sequences of 10-100 contiguous bases, or their complements, comprising a polymorphic position. The databases may also contain personal information of individuals originating from interviews, questionnaires or surveys as well as relevant medical information originating from doctors, physicians, dieticians, nutritionists or genetic counselors. In addition, the databases may comprise information regarding all types of diets, dietary components and dietary weight loss intervention programs (including composition, price, dosage, etc). It may even comprise information regarding which diet, dietary component and dietary weight loss intervention program is suitable and/or not suitable for an individual on the basis of its genetic profile. The databases may comprise information from one individual but also from a group of individuals (e.g. a specific population or subpopulation). The databases may be used in the methods and uses of the present invention. Typically, genetic data from an individual will be introduced into the computer system by means of electronic means, for example by using a computer. Next, the genetic data are compared to the data in the databases comprising information relating to genetic markers. On the basis of the comparison the likelihood of an individual to complete a dietary weight loss intervention program can be determined and, optionally, a suitable personalized diet can be advised. The invention also provides a computer program comprising program code means for performing all the above steps when said program is run on a computer. Also provided is a computer program product comprising program code means stored on a computer readable medium for performing the methods and uses of the invention when said program is run on a computer. A computer program product comprising program code means on a carrier wave that, when executed on a computer system, instruct the computer system to perform the above steps is additionally provided. The invention also provides an apparatus arranged to perform the above steps. The apparatus typically comprises a computer system, such as a PC. In one embodiment, the computer system comprises means for receiving genetic data from an individual, a module for comparing the data with a database comprising information relating to genetic markers, and means for determining on the basis of said comparison the likelihood that an individual will loose weight in a dietary weight loss intervention program and optionally even means to determine a suitable diet, dietary component or dietary weight loss intervention program for an individual. Access to the databases can be accomplished electronically, e.g. via a computer (PC or laptop), mobile phone, personal digital assistance, internet, handheld but the information in the databases can also be provided in paper form. People having access to the databases may be the individuals themselves, physicians, nutritionists, doctors, dieticians, and even restaurants and supermarkets. Access may be complete or limited to certain data only.

### EXAMPLES

To illustrate the invention, the following examples are provided. These examples are not intended to limit the scope of the invention.

### Example 1

### Introduction

Large genome-wide association studies (GWAS) have identified 32 gene loci in which single nucleotide polymorphisms (SNPs) are robustly, though weakly, related to body mass index (BMI), and fewer variants have been related to proxy measures of abdominal obesity. All these genetic variants have been related to body size measured at a single time point in adulthood, and have not addressed the influence on ability to loose weight, and the interaction with dietary factors.

Individuals differ greatly in weight loss response to energy restriction. Variation in adherence to a prescribed diet, and in physical activity and metabolic response determining energy efficiency may be involved. Twin studies indicate that genetic predisposition influence weight change response during underfeeding or overfeeding, but few studies have shown effects of various genotypes on weight loss response.

The optimal macronutrient composition of a diet for weight loss has been much debated.

The aim of this study was to examine the effect of the currently know panel of SNPs in gene loci with an established association with body size or waist measures, on changes in weight and waist circumference. In NUGENOB we investigated main genetic effects and interactions between SNPs in these gene loci and the randomized low-versus high-fat group, and also for reported changes in fat % and energy intake, in relation to weight loss and waist reduction. We aimed at replication of findings of strong effects in NUGENOB in another weight loss study (DiOGenes).

Design, Setting, and Participants: Randomized controlled trial of 771 obese adults with genotype data for each SNP in 617-642 of 648 completers. One SNP was replicated in another weight loss intervention of 934 obese adults with genotype and dietary intake data available in 590 of 803 completers.
Interventions: The trial was a 10-week 600 kcal/d energy-deficient diet of low or high fat (energy percent from fat, fat %, 20-25% and 40-45%). The other study used an 8-weeks diet of 880 kcal/d and 20 fat %, from which change in fat % intake from baseline was used for estimation of gene-dietary fat interaction effects.
Main outcome measures: Weight loss and waist reduction.
Results: In the trial, mean change in fat % intake was −12 in the low-fat group and four in the high-fat group, and in the other study −23 among those reducing fat % more than the median, and −12 among those reducing less. TFAP2B rs987237 genotype AA was associated with 1 kg (95% confidence interval, CI, 0.4; 1.6) more weight loss on the low-fat than on the high-fat diet, and GG genotype with 2.6 kg (95% CI, 1.1; 4.1) more weight loss on the high-fat than on the low-fat diet (P for interaction=0.00007). The other study showed a similar pattern for TFAP2B-dietary fat interaction.
Conclusions: Under energy restriction TFAP2B modifies the effect of dietary fat intake on weight loss.

### Participants and Methods

NUGENOB

Information on the NUGENOB trial is available through the Web (http://www.nugenob.com). Briefly, 771 obese men and women recruited to one of eight European centres were randomly assigned to a low- or high-fat diet with corresponding high or low carbohydrate content for ten weeks. Inclusion criteria were a BMI of at least 30 kg/m², and age 20-50 years. Participants were not included if they had experienced a weight change more than 3 kg within three months prior to the study, or if they reported other pre-identified characteristics that could influence the results. Dietary targets were an intake of 20-25% of energy derived from fat (fat %) in the low-fat group, and 40-45 fat % in the high-fat group. Both groups were prescribed a protein intake of 15 energy percent with no or minimal alcohol consumption, and the goal for energy intake was a daily energy deficit of approximately 600 kcal, calculated by (1.3×resting energy expenditure)−600. Dieticians instructed participants individually about how to reach the assigned diet, and also about how to weigh and record food intake. Weighed food recordings were requested from participants for one weekend day and two weekdays at baseline and at week ten of the intervention, and for one weekday at week two and five. The reported intake was analyzed locally on country-specific food databases. Besides the dietary intervention, participants were also asked to maintain their usual physical activity during the intervention.

Participants underwent a clinical examination, including anthropometry, at their local clinical center shortly before and after the intervention. Participants were instructed to avoid strenuous exercise and abstain from drinking alcohol three days prior to the examination, and to fast overnight and void the bladder before measurements. All variables of diet and anthropometry were checked for unrealistic values and outliers, and such values, two or less for each variable, were recoded to missing.

Gene Loci Selection and Genotyping

Gene loci included were those that had shown a consistent association with BMI, waist circumference, or waist-hip ratio in GWAS. In total 55 SNPs were included in the study. The FTO SNP was genotyped by Taqman allelic discrimination (KBioScience, Herts, UK), with a 96.9% genotyping success rate and a genotyping error rate of 0.27%. Genome-wide genotyping on the Illumine 317 k quad chip carried out at IntegraGen, Evry, France, covered another 23 SNPs in this study, with a 99.1% genotyping success rate. The remaining 31 SNPs were genotyped by KASPar SNP Genotyping (KBioscience, Hoddesdon, UK). Success rate was above 97% and an error rate was below 0.5% in 183 replicate samples. We used Fisher's exact test to evaluate Hardy-Weinberg equilibrium (HWE) for all SNPs.

Statistical Analysis

Drop-out rates by genotypes were compared by Pearson Chi-square statistics. The baseline BMI of completers and non-completers were compared by two-sample t-test.

We used linear regression to calculate main genotype effects, and gene-diet interactions, in relation to changes in weight and waist. The additive genetic effects and the sum of genotypes, each encoded 0, 1, or 2, equivalent to the number of obesity risk-alleles, were analyzed. Dietary factors investigated were the two randomized groups, and reported change in fat % and energy intake. We also investigated SNP interactions with the combination of low-fat diet and >2 unit increase in protein % intake because this group showed greater weight loss than the counterpart group in our previous study. Similar but weaker results to that of SNP-randomized fat group interactions suggested that the randomized groups mainly drove associations, so we omitted detailed analysis and presentation of these results.

Individual change in dietary intake was calculated as the difference between mean intake over three days at baseline, and the intake over five days during the intervention, with equal weight for intake at the first part versus end of intervention. Change in weight and waist circumference corresponded to the difference between levels at baseline and directly after the intervention. All regression models included change in weight or waist (wgt/wst) as dependent variable, and explanatory variables were genotype, baseline wgt/wst, sex, baseline wgt/wst×sex, age, and study center. Analyses of gene-diet interactions additionally included a product term of genotype (encoded 0, 1, 2) and the dietary factor of interest and also the dietary factor in single. Genetic effects on weight and waist change are denoted as effects on weight loss and waist reduction. All statistical tests were two-sided and were performed in Stata 9.2 (StataCorp, LP, College Station, 2007).

Replication in DiOGenes

The strongest effects observed in NUGENOB were replicated in the DiOGenes study (Diet, Obesity and Genes, www.diogenes-eu.org). The trial has been described in detail by Larsen et al. Briefly, DiOGenes is a dietary intervention study of obese adults undergoing two phases; an 8-weeks weight loss phase on a low-calorie diet, and 6-12 months weight maintenance phase on one of five diets. Data from the weight loss phase were used in the present study.

Study participants were men and women with a BMI between 27 and 45 kg/m², recruited to one of eight European centers. Exclusion criteria were characteristics that could potentially influence the results, such as more than 3 kg weight change within two months prior to the study and medications or certain diseases or disorders. Participants of the study recorded their baseline dietary intake during two weekdays and one weekend day. Weighed food recordings were applied with instructions and procedures similar to those in NUGENOB. The 8-weeks weight loss intervention consisted of a low-calorie Modifast diet (Nutrition et Santé). Four items per day were selected from various products, each containing 202-218 kcal. Participants were also allowed a daily intake of 200 g tomatoes, 125 g cucumber, and 50 g lettuce. The low-calorie diet provided approximately 880 kcal/d, with an energy-percent from fat of 20%, 54% from carbohydrates, and 26% from protein.

Anthropometric measurements were standardized across the study centers. Height was measured four weeks before the weight loss trial. Weight and waist circumference were measured 1-3 weeks before the trial and right after the intervention.

Genotyping of TFAP2B was performed on the Illumine 660 k quad chip carried out at the Centre National de Génotypage (CNG), Evry, France.

Statistical analysis was conducted as described for NUGENOB. Dietary fat in gene-diet interaction analyses was in DiOGenes assessed for change in fat % intake, calculated as the difference between reported baseline intake, and the fat proportion of the intervention diet. The inter-individual variation for calculated fat % change was therefore exclusively determined by baseline dietary intake. Median fat % change was used as cut-point for two groups of fat % change, which replaced the randomized fat groups in NUGENOB. Statistical models were identical in all other aspects.

Ethical Considerations

The NUGENOB and DiOGenes trials have obtained written informed consent from all study participants, and ethical approval from the ethical committee at each study center.

Results

NUGENOB

Out of 771 participants at start of the intervention, 648 (84%) completed it (FIG. 1). Completers and non-completers did not differ significantly in baseline BMI (P=0.8). One weight gainer with an extreme baseline BMI of 66 kg/m² and five participants with missing genotype data were excluded. For each of the 55 variants included in the study, genotype data were available in 617-642 participants (95-99% of completers). Genotype distributions were not related to drop-out rates except for ETV5 and FAIM2, which had more completers among homozygotes than among non-carriers of the obesity risk-allele (P=0.01 and 0.02 respectively), and for PTBP2, which showed the opposite pattern (P=0.03). Of completers with genotype data, 580 (90% of 642) participants had dietary intake data available, and they were included in gene-diet interaction analyses that included reported dietary intake.

Participants had a mean baseline BMI of 35.5 kg/m², and the mean weight was 96.8 kg in women and 110.6 kg in men (Table 1).

TABLE 1
Mean (SD) baseline level and change of body size by sex in 642 completers
with genotype data available, and dietary intake by randomized group in
580 completers with genotype data and dietary intake data available

| | Women (n = 481) | | Men (n = 161) | | All (n = 642) | |
|---|---|---|---|---|---|---|
| Variable | Baseline | Change¹ | Baseline | Change¹ | Baseline | Change¹ |
| Age (years) | 36.5 (7.9) | | 38.7 (7.6) | | 37.1 (7.9) | |
| Weight, kg | 96.8 (14.5) | −6.5 (3.2) | 110.6 (16.3) | −7.9 (3.8) | 100.3 (16.1) | −6.8 (3.4) |
| BMI, kg/m² | 35.8 (4.8) | −2.4 (1.2) | 34.7 (4.5) | −2.5 (1.2) | 35.5 (4.7) | −2.4 (1.2) |
| Waist circum- | 103.2 (12.0) | −5.9 (4.5) | 113.9 (11.4) | −7.7 (4.3) | 105.9 (12.7) | −6.3 (4.5) |
| ference, cm | | | | | | |

| | Low-fat diet (n = 298) | | High-fat diet (n = 282) | | All (n = 580) | |
|---|---|---|---|---|---|---|
| | Baseline | Change² | Baseline | Change² | Baseline | Change² |
| Energy intake, kcal/d | 2201 (691) | −652 (632) | 2194 (625) | −576 (534) | 2197 (659) | −615 (587) |
| Fat, E %³ | 36.7 (7.6) | −11.6 (8.2) | 37.0 (7.8) | 3.9 (9.0) | 36.9 (7.7) | −4.1 (11.6) |
| Fat, g | 90.9 (37.3) | −47.4 (34.8) | 91.2 (35.7) | −17.5 (33.2) | 91.1 (36.5) | −32.9 (37.2) |
| Carbohydrates, E %³ | 45.2 (8.6) | 11.5 (8.9) | 44.8 (8.7) | −2.9 (9.5) | 45.0 (8.6) | 4.5 (11.7) |
| Carbohydrates, g | 246.7 (86.4) | −27.2 (85.3) | 245.0 (80.2) | −75.8 (76.7) | 245.9 (83.4) | −50.9 (84.7) |
| Protein, E %³ | 16.3 (3.4) | 1.6 (3.6) | 16.3 (3.2) | 0.7 (3.6) | 16.3 (3.3) | 1.2 (3.6) |
| Protein, g | 87.7 (27.6) | −18.7 (26.0) | 88.4 (27.6) | −19.7 (23.9) | 88.1 (27.6) | −19.2 (25.0) |

E %, percent of total energy intake.
¹Difference between baseline and after the intervention.
²Difference between reported intake at baseline and during the intervention.
³Alcohol contributed with on average 1.8 E % (6.3 g) at baseline and the proportion reduced during the intervention.

Energy intake was reduced by on average 615 kcal/d in the study group. This was achieved by a reduction in absolute intake of macronutrients, with a greater decrease in fat intake in the low-fat group, and a greater decrease of carbohydrates in the high-fat group. On average, women lost on average 6.5 kg weight and 5.9 cm waist and men lost 7.9 kg weight and 7.7 cm waist during the intervention.

The distribution of genotypes, allele frequency, and outcome of HWE test for the 55 gene loci are reported in Table 2. All minor allele frequencies were larger than 0.05, and HWE tests were acceptable with only one SNP (PFKP rs6602024) showing a P-value lower than 0.1.

TABLE 2
Genotype information of the 55 gene loci included in the study

| | | | | Genotype | | | Hardy-Weinberg | |
|---|---|---|---|---|---|---|---|---|
| | | | Risk- | Non- | Hetero- | Homo- | Minor | P- |
| Gene | SNP | Trait | allele | carrier | zygote | zygote ¹ | allele freq | value ² |
| BDNF | rs4923461 | BMI | A | 32 | 210 | 385 | .22 | 6 |
| CADM2 | rs13078807 | BMI | G | 392 | 202 | 24 | .20 | 7 |
| CTNNBL1 | rs6013029 | BMI | T | 555 | 77 | 3 | .07 | 9 |
| ETV5 | rs9816226 | BMI | T | 9 | 192 | 423 | .17 | 01 |
| FANCL | rs887912 | BMI | T | 308 | 264 | 63 | .31 | 6 |
| FLJ35779 | rs2112347 | BMI | T | 71 | 289 | 271 | .34 | 6 |
| GNPDA2 | rs10938397 | BMI | G | 198 | 302 | 129 | .45 | 5 |
| GPRC5B | rs12444979 | BMI | C | 6 | 131 | 492 | .12 | 4 |
| KCTD15 | rs29941 | BMI | G | 53 | 280 | 288 | .31 | 2 |
| LRP1B | rs2890652 | BMI | C | 448 | 167 | 16 | .16 | 9 |
| LRRN6C | rs10968576 | BMI | G | 292 | 271 | 62 | .32 | 9 |
| MAF | rs1424233 | BMI | A | 170 | 299 | 151 | .48 | 4 |
| MAP2K5 | rs2241423 | BMI | G | 21 | 224 | 381 | .21 | 08 |
| MTCH2 | rs10838738 | BMI | G | 250 | 289 | 85 | .37 | 9 |
| MTIF3 | rs4771122 | BMI | G | 359 | 230 | 40 | .25 | 7 |
| NEGR1 | rs2568958 | BMI | A | 94 | 280 | 253 | .37 | 3 |
| NPC1 | rs1805081 | BMI | A | 89 | 292 | 244 | .38 | 9 |
| NUDT3 | rs206936 | BMI | G | 386 | 214 | 32 | .22 | 7 |
| PCSK1 | rs6235 | BMI | C | 368 | 217 | 45 | .24 | 1 |
| PFKP | rs6602024 | BMI | A | 511 | 110 | 15 | .11 | 003 |
| PRKD1 | rs11847697 | BMI | T | 569 | 66 | 0 | .14 | 4 |
| PRL | rs4712652 | BMI | A | 130 | 286 | 214 | .43 | 06 |
| PTBP2 | rs1555543 | BMI | C | 116 | 318 | 199 | .43 | 6 |
| PTER | rs10508503 | BMI | C | 5 | 93 | 526 | .08 | 7 |
| QPCTL | rs2287019 | BMI | C | 24 | 189 | 411 | .19 | 7 |
| RBJ | rs713586 | BMI | C | 172 | 300 | 158 | .49 | 2 |
| RPL27A | rs4929949 | BMI | C | 157 | 321 | 157 | .50 | 8 |
| SEC16B | rs10913469 | BMI | C | 430 | 174 | 22 | .17 | 4 |
| SH2B1 | rs7498665 | BMI | G | 220 | 271 | 126 | .42 | 01 |
| SLC39A8 | rs13107325 | BMI | T | 535 | 88 | 4 | .08 | 9 |
| TMEM160 | rs3810291 | BMI | A | 60 | 278 | 282 | .32 | 5 |
| TMEM18 | rs2867125 | BMI | G | 15 | 158 | 453 | .15 | 8 |
| TNNI3K | rs1514175 | BMI | A | 198 | 309 | 121 | .44 | .0 |
| ZNF608 | rs4836133 | BMI | A | 190 | 284 | 155 | .47 | 02 |
| FAIM2 | rs7138803 | BMI, | A | 244 | 295 | 86 | .37 | 8 |
| | | waist | | | | | | |
| FTO | rs9939609 | BMI, | A | 205 | 311 | 102 | .42 | 4 |
| | | waist | | | | | | |
| MC4R | rs12970134 | BMI, | A | 298 | 272 | 57 | .31 | 7 |
| | | waist | | | | | | |
| NRXN3 | rs10146997 | BMI, | G | 390 | 221 | 31 | .22 | .0 |
| | | waist | | | | | | |
| TFAP2B³ | rs987237 | BMI, | G | 430 | 186 | 26 | .19 | 3 |
| | | waist | | | | | | |
| MSRA | rs545854 | Waist | G | 462 | 161 | 14 | .15 | .0 |
| ADAMTS9 | rs6795735 | Waist-hip | C | 126 | 313 | 188 | .45 | 8 |
| | | ratio | | | | | | |
| CPEB4 | rs6861681 | Waist-hip | A | 320 | 261 | 53 | .29 | .0 |
| | | ratio | | | | | | |
| DNM3- | rs1011731 | Waist-hip | G | 235 | 283 | 102 | .39 | 3 |
| PIGC | | ratio | | | | | | |
| GRB14 | rs10195252 | Waist-hip | T | 110 | 283 | 230 | .40 | 2 |
| | | ratio | | | | | | |
| HOXC13 | rs1443512 | Waist-hip | A | 359 | 238 | 29 | .24 | 2 |
| | | ratio | | | | | | |
| ITPR2- | rs718314 | Waist-hip | G | 350 | 223 | 46 | .25 | 2 |
| SSPN | | ratio | | | | | | |
| LYPLAL1 | rs2605100 | Waist-hip | G | 62 | 262 | 303 | .31 | 6 |
| | | ratio | | | | | | |
| LYPLAL1 | rs4846567 | Waist-hip | G | 56 | 267 | 312 | .30 | 9 |
| | | ratio | | | | | | |
| LY86 | rs1294421 | Waist-hip | G | 96 | 281 | 263 | .37 | 1 |
| | | ratio | | | | | | |
| NFE2L3 | rs1055144 | Waist-hip | T | 413 | 191 | 23 | .19 | 9 |
| | | ratio | | | | | | |
| NISCH- | rs6784615 | Waist-hip | T | 2 | 66 | 567 | .06 | .0 |
| STAB1 | | ratio | | | | | | |
| RSPO3 | rs9491696 | Waist-hip | G | 168 | 292 | 164 | .50 | 1 |
| | | ratio | | | | | | |
| TBX15- | rs984222 | Waist-hip | G | 111 | 302 | 224 | .41 | 6 |
| WARS2 | | ratio | | | | | | |
| VEGFA | rs6905288 | Waist-hip | A | 122 | 325 | 191 | .45 | 4 |
| | | ratio | | | | | | |
| ZNRF3- | rs4823006 | Waist-hip | A | 110 | 315 | 211 | .42 | 7 |
| KREMEN1 | | ratio | | | | | | |

freq, frequency; BMI, body mass index.
¹ Homozygous for the obesity trait risk-allele.
² Fisher's exact test.
³Numbers in DiOGenes for the five last columns were: 435, 188, 21, .18, and .9.

Table 3 shows P-values for main effects and interactions with diet for 17 gene loci that reached nominal P-values below 0.05 for an association with weight loss or waist reduction. The most striking results were found for the SNP of TFAP2B (rs987237). This SNP modified the effect on weight loss by fat group with a nominal P-value for interaction of 0.00007, which also reached significance after correction for multiple testing of 55 SNPs (0.05/55=0.0009). In an additive gene-diet interaction model, homozygotes for the A allele lost 1 kg (95% confidence interval, CI, 0.4; 1.6) more weight on the low-fat than on the high-fat diet, whereas homozygotes for the G allele, i.e. the obesity risk-allele, lost 2.6 kg (95% CI, 1.1; 4.1) more weight on the high-fat than the low-fat diet (Table 4). A similar but weaker pattern was observed for waist reduction (P for interaction=0.03). TFAP2B was also directly related to weight loss (P=0.04) with a 0.5 kg (95% CI, 0.1; 0.9) greater weight loss per additional G allele.

TABLE 3
P-value for main genotype effect and interaction between genotype and
diet in relation to weight loss and waist reduction for 17 gene loci with a P-value <.05¹
for one or more associations

| | | All participants | | Participants with dietary | |
|---|---|---|---|---|---|
| | | (n = 617-642) | | data (n = 559-580) | |
| | | | Gene × low-/ | Gene × | Gene × |
| | Phenotype | Genotype | high-fat | change⁵ in | change⁵ in |
| Gene² | change | effect³ | diet⁴ | fat % intake⁴ | energy intake⁴ |
| CTNNBL1 | Weight | .4 | .1 | .7 | .8 |
| | Waist | .5 | .0003 | .05 | .2 |
| FANCL | Weight | .2 | .6 | .8 | .03 |
| | Waist | .5 | .5 | .9 | .9 |
| GPRC5B | Weight | .2 | .1 | .046 | .2 |
| | Waist | .4 | .5 | .5 | .6 |
| LRRN6C | Weight | .3 | .01 | .1 | .5 |
| | Waist | .9 | .02 | .4 | .9 |
| MAF | Weight | .7 | .04 | .1 | .09 |
| | Waist | .4 | .1 | .2 | .05 |
| MAP2K5 | Weight | .9 | 1.0 | .3 | .9 |
| | Waist | .4 | .7 | .9 | .03 |
| MTIF3 | Weight | .1 | .4 | .3 | .6 |
| | Waist | .04 | .8 | .5 | .7 |
| NPC1 | Weight | .7 | .04 | .2 | 1.0 |
| | Waist | .1 | .008 | .9 | .6 |
| SLC39A8 | Weight | .7 | .4 | .8 | .9 |
| | Waist | .3 | .2 | .03 | .7 |
| ZNF608 | Weight | .06 | .6 | .9 | .1 |
| | Waist | .01 | .5 | .6 | .7 |
| MC4R | Weight | .09 | .3 | .4 | .049 |
| | Waist | .5 | .9 | .9 | .2 |
| TFAP2B | Weight | .04 | .00007 | .03 | .8 |
| | Waist | .1 | .03 | .2 | .5 |
| ADAMTS9 | Weight | .9 | .2 | .2 | .6 |
| | Waist | .2 | .04 | .1 | 1.0 |
| DNM3-PIGC | Weight | .8 | .6 | .2 | .02 |
| | Waist | .3 | .3 | .8 | .2 |
| LY86 | Weight | .02 | .4 | .6 | .2 |
| | Waist | .1 | .7 | .7 | .3 |
| RSPO3 | Weight | .3 | .1 | .5 | .01 |
| | Waist | .3 | .045 | .2 | .3 |
| VEGFA | Weight | .001 | .2 | .4 | .9 |
| | Waist | .03 | .6 | .7 | .9 |

²See SNP information in Table 2.
³P-value for the additive effect of genotype adjusted for baseline weight or waist (weight/waist), sex, baseline weight/waist × sex, age, and center.
⁴P-value for the product term of genotype and the dietary factor of interest, adjusted for baseline weight/waist, sex, baseline weight/waist × sex, age, center, genotype, and the dietary factor of interest.
⁵Difference between reported intake at baseline and during the intervention.

TABLE 4
Weight and waist change (95% CI)* in the high-fat versus low-fat group in
NUGENOB, by genotype:

| | | | | P-value for |
|---|---|---|---|---|
| | | | | gene-fat group |
| | AA | AG | GG | interaction |
| Weight change, kg | 1 (0.4; 1.6) | −0.8 (−1.5; −0) | −2.6 (−4.1; −1.1) | 0.00007 |
| Waist change, cm | 0.9 (0.1; 1.7) | −0.4 (−1.4; 0.6) | −1.7 (−3.8; 0.3) | 0.03 |

*Numbers relate to the difference in weight loss so positive numbers indicate the amount greater weight loss in the low-fat group (e.g. 0.99 kg, 95% CI: 0.40 to 1.58, in the first cell) compared to the high-fat group, and negative numbers indicate a greater weight loss (and amount) in the high-fat group.

Four other gene loci showed P-values between 0.0003 and <0.01 for a main effect or interaction with diet, in relation to weight loss or waist reduction (eTable 1). These were main effects on weight loss by VEGFA, and a gene-fat group interaction in relation to waist reduction for CTNNBL1 and NPC1.

The gene score (sum of all genotypes) showed no main effect or interaction with diet in relation to outcomes (P≧0.09).

Replication of TFAP2B-fat interaction analyses in DiOGenes In total 934 participants started the weight loss trial in DiOGenes, and 803 (86%) completed it. Among those, genotype data were available in 639 participants, of which 590 participants had reported their dietary intake before the intervention. Completers did not differ significantly from non-completers with respect to BMI (P=0.7), or TFAP2B genotype distribution (P=0.4).

Characteristics of DiOGenes participants are shown in Table 5. Whereas the range for change in fat % intake in NUGENOB was broad and largely related to randomized group, fat intake among DiOGenes participants reduced much both in terms of absolute intake (mean: −74.1 g/d, range: −251.2 to −0.1 g) and fat % (mean: −17.3, range −43.7 to 1.7). The mean energy intake reduction of 1351 kcal/d in DiOGenes was more than double the reduction in NUGENOB, and weight loss and waist reduction was greater. In contrast to NUGENOB where we previously found an association between reduced reported energy intake and weight loss (0.08 kg weight loss per 100 kcal/d reduction, P<0.001), the association in DiOGenes based on self-reported baseline dietary intake and the energy content of the low-calorie diet, was weak (0.02 kg per 100 kcal/d, P=0.3).

Analysis of TFAP2B combined with a fat % reduction above or lower than the median of 16.7 fat % showed similar patterns for associations with weight loss and waist reduction to those in NUGENOB, but statistical evidence for interaction was weak (P=0.4) (Table 6). There were no main effects of TFAP2B on weight loss (P=0.8) or waist reduction (P=0.6).

TABLE 5
Mean (SD) baseline level and change of body size by sex in 639 completers in DiOGenes in
whom TFAP2B was successfully genotyped, and dietary intake in groups split by median change
in fat % intake in 590 completers with TFAP2B data and dietary intake data available

| | Women (n = 415) | | Men (n = 224) | | All (n = 639) | |
|---|---|---|---|---|---|---|
| Variable | Baseline | Change¹ | Baseline | Change¹ | Baseline | Change¹ |
| Age, years | 41.0 (6.3) | | 43.0 (5.5) | | 42.0 (6.1) | |
| Weight, kg | 94.9 (15.4) | −9.9 (3.0) | 109.5 (17.3) | −12.8 (4.1) | 100.0 (17.5) | −10.9 (3.7) |
| BMI, kg/m² | 34.5 (4.9) | −3.7 (1.0) | 34.4 (4.6) | −4.0 (1.2) | 34.5 (4.8) | −3.8 (1.1) |
| Waist circum- | 104.3 (11.8) | −9.1 (4.7) | 114.6 (12.4) | −11.1 (4.3) | 107.9 (13.0) | −9.8 (4.7) |
| ference, cm | | | | | | |

| | >16.7 fat % decrease | | <16.7 fat % decrease² | | | |
|---|---|---|---|---|---|---|
| | (n = 295) | | (n = 295) | | All (n = 590) | |
| | Baseline | Change³ | Baseline | Change³ | Baseline | Change³ |
| Energy intake, kcal/d | 2307 (774) | −1427 (774) | 2154 (778) | −1274 (777) | 2231 (779) | −1351 (779) |
| Fat, E %⁴ | 43.5 (5.1) | −23.0 (5.1) | 32.0 (3.8) | −11.6 (3.8) | 37.8 (7.3) | −17.3 (7.3) |
| Fat, g | 111.1 (40.5) | −91.1 (40.5) | 77.4 (31.3) | −57.4 (31.3) | 94.1 (39.8) | −74.1 (39.8) |
| Carbohydrates, E %⁴ | 38.0 (6.2) | 15.7 (6.2) | 47.6 (7.2) | 6.0 (7.2) | 42.8 (8.3) | 10.8 (8.3) |
| Carbohydrates, g | 218.1 (77.6) | −100.1 (77.6) | 257.2 (100.3) | −139.2 (100.3) | 237.5 (91.7) | −119.5 (91.7) |
| Protein, E %⁴ | 17.1 (4.0) | 8.8 (4.0) | 17.0 (4.5) | 8.9 (4.5) | 17.1 (4.3) | 8.8 (4.3) |
| Protein, g | 96.5 (34.5) | −39.5 (34.5) | 88.3 (30.3) | −31.3 (30.3) | 92.4 (32.7) | −35.4 (32.7) |

E %, percent of total energy intake.
¹Difference between baseline and after the intervention.
²One participant reported an increased (1.7 units) fat % intake.
³Difference between reported intake at baseline and the standardised low-calorie diet of 880 kcal/day, including macronutrients: fat, 20 g, 20 E %; carbohydrates, 118 g, 54 E %; and protein, 57 g, 26 E %.
⁴Alcohol contributed with on average 2.3 E % (7.9 g) at baseline and no alcohol was allowed during the intervention.

TABLE 6
Weight and waist change (95% CI) in the group of <16.7 unit reduction (less
than median reduction) in energy percent intake from fat (fat %) versus the group of
>16.7 fat % reduction in DiOGenes, by genotype:

| | | | | P-value for |
|---|---|---|---|---|
| | | | | gene-fat group |
| | AA | AG | GG | interaction |
| Weight change, kg | 0.1 (−0.5; 0.7) | −0.3 (−1.; 0.5) | −0.7 (−2.2; 0.9) | 0.4 |
| Waist change, cm | 0. (−0.9; 0.9) | −0.6 (−1.8; 0.6) | −1.2 (−3.6; 1.2) | 0.4 |

### Comment

In this study of weight loss in obese men and women on a 10-weeks energy restricted diet low or high in fat to carbohydrate intake, we investigated main effects and gene-diet interactions of 55 gene loci with an established association with BMI or waist measures at genome-wide significant levels. We found that the TFAP2B gene locus rs987237 clearly modified the effect of a high versus low-fat diet on weight loss. Whilst non-carriers of the obesity risk-allele lost more weight on the low-fat diet, the opposite was shown among homozygotes of the obesity risk-allele. These results showed a similar, pattern in another weight loss study of a low-calorie diet for eight weeks when comparing participants with a small versus large reduction of fat % intake. The similar pattern of results from these different studies indicates that under energy restriction, TFAP2B may influence the effect of dietary fat on weight loss.

Several factors may underlie the much weaker associations observed in the replicated analysis. Analyses in NUGENOB were performed in line with the original design; two randomized groups of low or high fat % intake were compared. Besides the controlling of unknown confounders owing to randomization, the two groups in NUGENOB were also more diverse in their changed fat % intake than were the constructed groups with different decreases in dietary fat % intake in DiOGenes. Moreover, we lacked information on dietary intake during the intervention in DiOGenes, so dietary change calculations assumed complete adherence to the prescribed diet. This assumption is likely not to be entirely met, as indicated by the lack of association between calculated energy intake reduction and weight loss in DiOGenes. Given these limitations to enable replication of the findings in NUGENOB, the similar pattern observed in DiOGenes is quite noteworthy. Many of the same limitations in DiOGenes confer also to NUGENOB for investigation of change in fat % and energy intake. Misclassification of self-reported dietary intake, small variation in exposure or small differences between compared groups, and confounding issues may have obscured the finding of further strong gene-diet interactions in NUGENOB, and a clearer replication of TFAP2B-dietary fat interaction in DiOGenes.

Other limitations of the study are the incomplete data due to drop-outs of study participants, and incomplete genotyping and dietary intake reports. Around 15% of study participants did not complete the trials; however, completers and non-completers did neither differ in baseline BMI, nor in overall genotype distribution. Dietary intake data were incomplete in both studies, but were based on weighed food records over several days that, despite its weaknesses owing to self-report, is frequently used as gold standard method for assessment of dietary intake.

The major strength of the study is the relatively large sample size of two well-controlled intervention studies of similar participant characteristics, allowing replication of analyses for highly significant findings. Replication is crucial to avoid spurious chance findings emerging from multiple testing. Although analyses in DiOGenes did not replicate the strong findings in NUGENOB, the patterns of results were consistent.

The mechanisms whereby TFAP2B could modify the effect of dietary fat intake on weight loss are unclear. It is also unclear whether TFAP2B indeed interacts with dietary fat, or rather with carbohydrates, or the fat-carbohydrate ratio. Speculatively, individuals of different TFAP2B genotype and gene expression metabolise fat or other macronutrients differently, and thus, respond differently to dietary change of macronutrient composition.

In conclusion, this study of obese men and women showed a clear interaction between TFAP2B and a diet low or high in fat, on weight loss under energy restriction. A similar pattern for interaction between TFAP2B and dietary fat intake was shown in another weight loss study of similar participant characteristics. Results of our study strongly encourage further examination of the role of TFAP2B and macronutrients in weight loss.

1. A method for assessing whether an individual will loose more weight when consuming a low fat/high carbohydrate diet or when consuming a high fat/low carbohydrate diet, the method comprising the step of determining the genotype of the single nucleotide polymorphism rs987237 in the TFAP2B gene, wherein:
(i) the presence of two A allelic forms of the SNP rs987237 is indicative of an increased likelihood that the individual looses more weight on a low fat/high carbohydrate diet compared to a high fat/low carbohydrate diet and,
(ii) the presence of AG or GG allelic forms of the SNP rs987237 is indicative of an increased likelihood that the individual looses more weight on a high fat/low carbohydrate diet compared to a low fat/high carbohydrate diet.

2. A method according to claim 1, wherein the individual is overweight or obese.

3. A method according to claim 1, wherein the high fat/low carbohydrate or low fat/high carbohydrate diet is a hypo-energetic diet.

4. A method for making a dietary weight-loss intervention program for an individual, the method comprising the steps of:
(i) identifying the individual's genotype for the single nucleotide polymorphism rs987237 in the TFAP2B gene,
(ii) using a low fat/high carbohydrate diet in the making of a dietary weight-loss intervention program suitable for treating an individual which has been identified as having two A allelic forms of the single nucleotide polymorphism rs987237 in the TFAP2B gene,
and using a high fat/low carbohydrate diet in the making of a dietary weight-loss intervention program suitable for treating an individual which has been identified as having AG or GG allelic forms of the single nucleotide polymorphism rs987237 in the TFAP2B gene.

5. A method of claim 4, wherein the individual is overweight or obese.
